(19) United States
(12) Patent Application Publication
(10) Pub. No.: US 2023/0056606 A1
(43) Pub. Date: Feb. 23, 2023

(54) Cosmetic Skin Cream and Medicine

(71) Applicant: Robert H. Schiestl, Los Angeles, CA (US)

(72) Inventor: Robert H. Schiestl, Los Angeles, CA (US)

(21) Appl. No.: 17/398,960

(22) Filed: Aug. 10, 2021

(51) Int. Cl. A61K 8/49; A61K 8/92; A61K 8/9794; A61K 8/64; A61K 8/06; A61Q 19/00

(52) U.S. Cl. CPC A61K 8/4926; A61K 8/922; A61K 8/9794; A61K 8/925; A61K 8/64; A61K 8/062; A61Q 19/00; A61K 2800/43; A61K 2800/5922

(57) ABSTRACT

Embodiments of the present disclosure recite medicated cosmetic skin creams containing Yel002, methods of manufacturing said creams, and methods of using said skin creams.

### BACKGROUND

The present disclosure relates generally to a novel skin cream and medicine composition and more particularly to a skin cream containing the compound Yel002 and skin beneficial substances.

In general, exposure to UV, ionizing radiation, radioactive iodine, environmental pollution, cigarette smoke, and chemical carcinogenic agents exert severe stress on the skin, often times leading to accelerated aging of the skin such as the appearance of wrinkles, sagging skin, discoloration, inflammation, itching, and other cosmetically undesirable effects, as well as more health-related issues such as skin cancer. Additionally, many people suffer from skin-related issues, including psoriasis, keratosis, acne, eczema, wounds, incisions, precancerous skin lesions, inflammation, skin pains and pains under the skin, as well as deep tissue diseases. Additionally, almost all surgeries make incisions in the skin which must heal.

The compound Yel002, shown below, has been discovered to be effective in mitigating tissue damage induced by an agent:

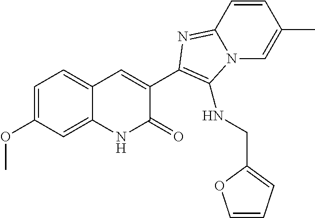 [SMILES: COc1ccc2cc(-c3nc4ccc(C)cn4c3NCc3ccco3)c(=O)[nH]c2c1]

The effectiveness and manufacture of Yel002 was disclosed in U.S. Pat. No. 9,045,474, granted on Jun. 2, 2015, and titled COMPOUNDS AND COMPOSITIONS FOR MITIGATING TISSUE DAMAGE AND LETHALITY, the entirety of which is incorporated herein by reference.

In order to overcome the skin problems described above, there are many cosmetic compositions on the market for use on the skin. However, there is a need for skin treatments that are economical, effective, and simple to apply, such as in a skin cream, that are capable of smoothing and firming the skin, while at the same time treating skin diseases such as psoriasis and keratosis and protecting against skin damage resulting from UV and pollutant exposure, inflammation, wounds, incisions, as well as other medical diseases and pains on and below the skin.

The embodiments described below address the above-mentioned problems and needs.

### BRIEF SUMMARY

In accordance with one embodiment of the present disclosure, there is contemplated a skin cream comprising Yel002. In certain embodiments, the skin cream may further include at least one oil. More generally, the skin cream may contain between 0.2-5% Yel002, between 0.5-4.0% ginger oil, between 3-30% aloe vera gel, between 1-10% bees wax, between 3-40% cacao butter, between 50-90% avocado oil, and between 0.5%-3.0% Cosphaderm. In one specific embodiment, the skin cream may contain approximately 1% Yel002, approximately 2% ginger oil, approximately 9% aloe vera gel, approximately 2.5% bees wax, approximately 7.5% cacao butter, approximately 77% avocado oil, and approximately 1.4% Cosphaderm. Other embodiments may further include between 0.005-0.5% human recombinant albumin.

Another embodiment of the present disclosure contemplates a method of manufacturing a skin cream. In this embodiment, the skin cream is made by disposing a first container containing avocado oil in a water bath. The water bath is heated to approximately 70-90 C. Next, bees wax and cacao butter is dissolved into the first container. Next, aloe vera gel is introduced into a second container. Then, the contents of the first container are slowly poured into the second container, while the second container is mixed with a fast mixer to obtain a homogenous emulsion. Finally, Yel002, ginger oil and Cosphaderm is mixed into the second container until a homogenous emulsion is formed.

Yet another embodiment of the present disclosure contemplates a method of treating a skin condition by applying a skin cream containing Yel002 to the skin condition. Examples of skin conditions that may be treated under this embodiment include keratosis, psoriasis, inflammation of the skin, skin wrinkles, discoloration, lipomas, scarring, sagging skin, acne, precancerous skin lesions, incisions after surgery, and sunburn. In this method, the skin cream may include at least one oil. For example, the skin cream may contain between 0.2-5% Yel002, between 0.5-4% ginger oil, between 0.5 to 3% Cosphaderm, between 3-30% aloe vera gel, between 1-10% bees wax, between 3-40% cacao butter, and between 50-90% avocado oil. In a specific embodiment of the method, the skin cream may contain approximately 1% Yel002 approximately 2% ginger oil, approximately 1.4% Cosphaderm, approximately 9% aloe vera gel, approximately 2.5% bees wax, approximately 7.5% cacao butter, and approximately 77% avocado oil. The skin cream may also be used to provide pain relief to a user having pain on the skin or up to about 1.5 inches below the skin. This pain relief is achieved by applying a skin cream containing Yel002 to the location of pain.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

A new combination of ingredients, including a radiation mitigator, namely, Yel002, results in a cosmetic cream that provides protection against many types of skin damage. The cosmetic cream further acts to heal skin and under-the-skin diseases and pains. Yel002, shown below, is one active ingredient in the skin cream.

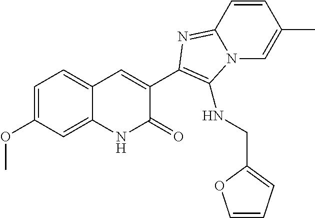 [SMILES: COc1ccc2cc(-c3nc4ccc(C)cn4c3NCc3ccco3)c(=O)[nH]c2c1]

The skin cream may include other cosmetic components known in the field. In a particular embodiment, the skin cream is made up of a combination of Yel002, ginger oil, aloe vera gel, bees wax, cacao butter, avocado oil and Cosphaderm. For example, one embodiment of the skin cream contains between 0.2-5% Yel002, between 0.5-4% ginger oil, between 0.5-3% Cosphaderm, between 3-30% aloe vera gel, between 1-10% bees wax, between 3-40% cacao butter, and between 50-90% avocado oil. More particularly, one formulation contains about 1% Yel002, about 2% ginger oil, 1.4% Cosphaderm, about 9% aloe vera gel, about 2.5% bees wax, about 7.5% cacao butter, and about 77% avocado oil. Other embodiments include 0.005-0.5% human recombinant albumin.

Each of the cosmetic components disclosed above contribute to the improved properties of the skin cream and medicine described herein and is present in a quantity sufficient to increase the smoothness and firmness of the skin to which the skin cream is applied, as well as providing protection against environmental pollutants and UV exposure. Further, application of the skin cream makes the skin glow and is anti-inflammatory and anti-aging. Additionally, these components in the skin cream are useful in treating skin conditions, such as psoriasis, keratosis, eczema, acne and the like. However, it is contemplated that other suitable oils or beneficial substances may be utilized, and other percentages of the components may be utilized. Additionally, the skin cream may further include other cosmetic components to add additional benefits to the skin cream, for example, but not limited to, emulsifiers, antioxidants, preservatives, solvents, thickeners, fragrances, colorants, human recombinant albumin, and the like. While it is common for skin creams within the art to be comprised of 70-80% aqueous solutions, the novel skin cream disclosed herein is made up of approximately 75% oils.

The cosmetic skin cream described herein provides numerous benefits to the skin of the user, but notably the cream makes the user's skin smoother and firmer. The skin cream described herein also has anti-inflammatory and anti-aging (human keratinocytes live 15% longer with the application of the active ingredient Yel002) properties and prevents wrinkles with continued use. The cream also beautifies the skin and makes it glow. Furthermore, the skin cream described herein has antibacterial and antifungal properties which serves to reduce the load on the immune system. In addition, rather than suppressing it, application of the cream redirects the immune system to be anti-inflammatory in relieving the inflammation in joints caused by arthritis. Also, the skin cream is effective in healing the pain and the skin wounds and incisions from surgery each at a rate 7× faster than without the cream.

With use, the skin cream described herein has been shown and/or is believed to protect the skin against ionizing radiation, UV, chemical carcinogens, chemical warfare agents, radioactive iodine, and cigarette smoke. Notably, the skin cream described herein has also been shown to heal psoriasis, keratosis, eczema, inflammation, of the face and toes, acne, precancerous skin lesions, sunburn, scars, discolored skin patches, to stop itching from insect and tick bites, and help prevent skin cancer. Unlike the use of cyclosporine to treat psoriasis, the skin cream as discussed above does not have immuno-suppressant effects. Also, the skin cream does not have the deleterious effect of disrupting DNA synthesis as occurs from use of the chemotherapy drug fluorouracil to treat keratosis. In particular, initial results with use of the cream described herein show improvement for ten patients from keratosis, three patients from psoriasis, two patients from eczema, one patient from inflammation of the face, three patients from inflammation of the toes, five patients from skin wrinkling, one patient from sunburn, two patients from scarring, three patients from discolored patches, one patient from precancerous lesions, and three patients from itching due to insect and tick bites. Additionally, more than 600 people saw their skin beautified and glowing from use of the skin cream. Some saw their hair look more shiny and healthy when using the cream on their hair. As such, the skin cream has applications throughout the human and animal body.

The skin cream also has deep tissue effects. It is well known that NSAIDS like ibuprofen are anti-inflammatory and analgesic. The skin cream disclosed herein is highly anti-inflammatory and thus also acts as a natural pain killer. It has been found that with use, the skin cream heals skin pains and pains up to about 1.5 inches below the skin (which is approximately 95% of all pains) after 1-3 weeks by applying the skin cream 1-5 times a day in a very thick layer, which is absorbed into the skin in about 1-20 minutes. Also, application of the skin cream is effective in eliminating pain at a rate 7× faster than without the cream. It has been observed that neck pains, knee pains, back pains, wrist and hand pains, inflamed tendons, rib and leg pains have been healed by such use of the skin cream. It has also been noted to heal whiplash syndrome, cysts, abscesses, arthritis, and lipomas. In particular, early results show relief from pain in the neck for two patients, in the knee for two patients, in the back for one patient, in the wrist and hands for three patients, in inflamed tendons for one patient, in the leg for four patients, along with relief from whiplash syndrome for one patient, cysts and abscess each for one patient, arthritis for three patients, and lipomas for two patients.

While the skin cream described herein may be formulated in suitable manners as is known in the art, it has been found to be effective to manufacture the skin cream by mixing the oils together in a heated water bath, and to then mix the aloe vera gel with a fast mixer and slowly add the oils to generate a smooth emulsion, and finally mix in the remaining ingredients.

The above description is given by way of example, and not limitation. Given the above disclosure, one skilled in the art could devise variations that are within the scope and spirit of the invention disclosed herein, including adjusting the proportion of the components within the composition. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A skin cream comprising Yel002.

2. The skin cream of claim 1 further comprising at least one oil.

3. The skin cream of claim 1 comprising:
0.2-5% Yel002;
0.5-4% ginger oil;
0.5-3% Cosphaderm;
3-30% aloe vera gel;
1-10% bees wax;
3-40% cacao butter; and
50-90% avocado oil.

4. The skin cream of claim 3 comprising:
approximately 1% Yel002;
approximately 2% ginger oil;
approximately 1.4% Cosphaderm;
approximately 9% aloe vera gel;
approximately 2.5% bees wax;
approximately 7.5% cacao butter; and
approximately 77% avocado oil.

5. The skin cream of claim 3 further comprising 0.005-0.5% human recombinant albumin.

6. A method of manufacturing a skin cream, said method comprising:
a) disposing a first container containing avocado oil in a water bath, wherein the water bath is heated to approximately 70-90 C;
b) dissolving bees wax and cacao butter into the first container of step a);
c) introducing aloe vera gel into a second container;
d) slowly pouring the contents of the first container obtained from step b) into the second container, while the second container is mixed with a fast mixer to obtain a homogenous emulsion; and
e) mixing Yel002, ginger oil and Cosphaderm into the second container containing the composition of step d) until a homogeneous emulsion is formed.

7. A method of treating a skin condition, said method comprising applying a skin cream containing Yel002 to the skin condition.

8. The method of claim 7, wherein the skin condition is keratosis.

9. The method of claim 7, wherein the skin condition is psoriasis.

10. The method of claim 7, wherein the skin condition is inflammation of the skin.

11. The method of claim 7, wherein the skin condition is skin wrinkles.

12. The method of claim 7, wherein the skin condition is discoloration.

13. The method of claim 7, wherein the skin condition is scarring.

14. The method of claim 7, wherein the skin condition is sagging skin.

15. The method of claim 7, wherein the skin condition is acne.

16. The method of claim 7, wherein the skin condition is a precancerous skin lesion.

17. The method of claim 7, wherein the skin condition is sunburn.

18. The method of claim 7, wherein the skin condition is arthritis.

19. The method of claim 7, whereas the skin condition is skin incisions during surgery.

20. The method of claim 7, whereas the skin condition is whiplash syndrome.

21. The method of claim 7, whereas the skin condition is abscess.

22. The method of claim 7, whereas the skin condition is skin pain.

23. The method of claim 7, wherein the skin cream further comprises at least one oil.

24. The method of claim 7, wherein the skin cream comprises:
0.2-5% Yel002;
0.5-4% ginger oil;
0.5-3% Cosphaderm,
3-30% aloe vera gel;
1-10% bees wax;
3-40% cacao butter; and
50-90% avocado oil.

25. The method of claim 21, wherein the skin cream comprises:
approximately 1% Yel002;
approximately 2% ginger oil;
approximately 1.4% Cosphaderm
approximately 9% aloe vera gel;
approximately 2.5% bees wax;
approximately 7.5% cacao butter; and
approximately 77% avocado oil.
